(19) United States
(12) Patent Application Publication
(10) Pub. No.: US 2021/0361228 A1
(43) Pub. Date: Nov. 25, 2021

(54) Sleep monitoring device and method

(71) Applicant: REALTEK SEMICONDUCTOR CORPORATION, Hsinchu (TW)

(72) Inventor: AI-HUA BIAN, Suzhou City (CN)

(21) Appl. No.: 17/319,123

(22) Filed: May 13, 2021

(30) Foreign Application Priority Data
May 19, 2020 (CN) 202010424402.X

(51) Int. Cl. A61B 5/00; A61B 5/11

(52) U.S. Cl. CPC A61B 5/4809; A61B 5/1118

(57) ABSTRACT

The present invention discloses a sleep monitoring device. A motion detection circuit detects a human body motion to generate a detection signal. A processing circuit retrieves and executes computer executable commands from a storage circuit to execute a sleep monitoring method including steps outlined below. The detection signal is received from the motion detection circuit. An activity amount, in a basic time duration, is accumulated based on a total time length that an activity strength of the detection signal is larger than a predetermined value. A statistics is generated based on the activity amount of each of the basic time duration for a plurality of different time ranges, to generate a total activity amount. Whether the total activity amount exceeds one of a plurality of activity threshold values is determined to generate an activity scale determination result. A sleep status is determined according to the activity scale determination result.

### BACKGROUND OF THE INVENTION

### 1. Field of the Invention

The present invention relates to a sleep monitoring device and a sleep monitoring method.

### 2. Description of Related Art

Besides basic functions of communication and time inquiry, wearable electronic devices provide more and more functions. In functions related to health monitoring, sleep monitoring function that detects the sleep status of a user is a common one.

Performing sleep monitoring function relies on detecting the activity amounts using the sensors for a prolonged period of time. However, many sleep monitoring technologies requires accumulating a huge amount of data, and as a result a large amount of storage and processing are utilized by the sleep monitoring operation of such electronic devices. Further, these sleep monitoring technologies determine sleep status solely based on activity amounts, and thus tiny movements such as those generated during sleeping or quiet reading can not be distinguished.

### SUMMARY OF THE INVENTION

In consideration of the problem of the prior art, an object of the present invention is to supply a sleep monitoring device and a sleep monitoring method.

The present invention discloses a sleep monitoring device that includes a motion detection circuit, a storage circuit and a processing circuit. The motion detection circuit is configured to detect a human body motion to generate a detection signal accordingly. The processing circuit is electrically coupled to the motion detection circuit and the storage circuit, and is configured to retrieve and execute computer executable commands to execute a sleep monitoring method that includes the steps outlined below. The detection signal is received from the motion detection circuit. An activity amount is accumulated, in each of basic time durations, based on a total time length, that an activity strength of the detection signal is larger than a predetermined value. For each of the basic time durations as a target basic time duration, statistics is generated based on the activity amount of all the basic time durations in different time ranges around the target basic time duration having different lengths, to generate total activity amounts of the time ranges corresponding to the target basic time duration. For each of the basic time durations, whether each of the total activity amounts exceeds one of a plurality of activity threshold values is determined to generate an activity scale determination result, wherein each of the activity threshold values corresponds to one of the time ranges. A sleep status is determined according to the activity scale determination result.

The present invention also discloses a sleep monitoring method used in a sleep monitoring device that includes the steps outlined below. A human body motion is detected by a motion detection circuit to generate a detection signal accordingly. The detection signal from the motion detection circuit is received by a processing circuit. An activity amount is accumulated, in each of basic time durations, based on a total time length, that an activity strength of the detection signal is larger than a predetermined value by the processing circuit. For each of the basic time durations as a target basic time duration, statistics is generated based on the activity amount of all the basic time durations in different time ranges around the target basic time duration having different lengths, to generate total activity amounts of the time ranges corresponding to the target basic time duration by the processing circuit. For each of the basic time durations, whether each of the total activity amounts exceeds one of a plurality of activity threshold values is determined to generate an activity scale determination result by the processing circuit, wherein each of the activity threshold values corresponds to one of the time ranges. A sleep status is determined according to the activity scale determination result by the processing circuit.

These and other objectives of the present invention will no doubt become obvious to those of ordinary skill in the art after reading the following detailed description of the preferred embodiments that are illustrated in the various figures and drawings.

### BRIEF DESCRIPTION OF THE DRAWINGS

FIG. 1 illustrates a block diagram of a sleep monitoring device according to an embodiment of the present invention.

FIG. 2 illustrates a diagram of the detection signal in a basic time duration according to an embodiment of the present invention.

FIG. 3 illustrates a flow chart of a sleep monitoring method according to an embodiment of the present invention.

### DETAILED DESCRIPTION OF THE PREFERRED EMBODIMENTS

An aspect of the present invention is to provide a sleep monitoring device and a sleep monitoring method to efficiently decrease the storage amount and operation amount required by the sleep monitoring operation and increase the accuracy of the sleep monitoring operation.

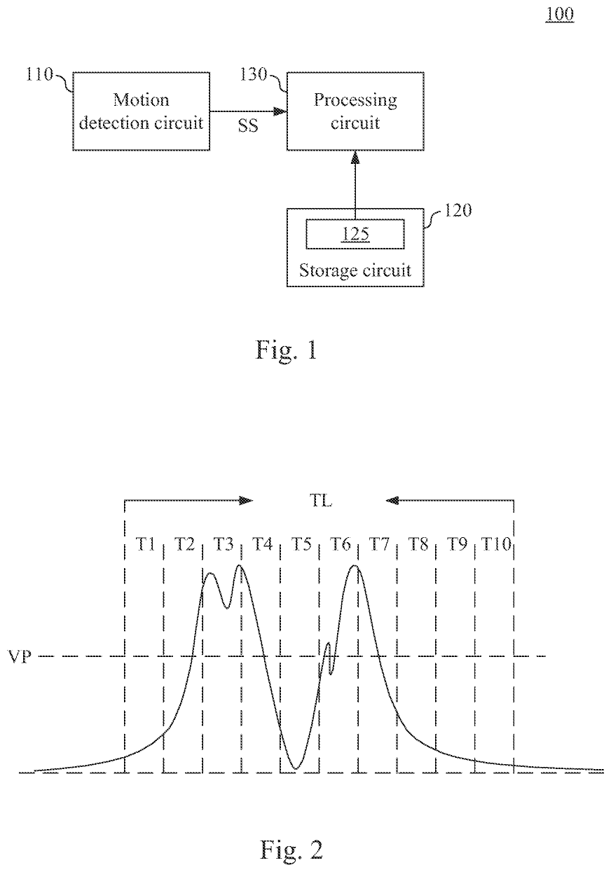

Reference is now made to FIG. 1. FIG. 1 illustrates a block diagram of a sleep monitoring device 100 according to an embodiment of the present invention. The sleep monitoring device 100 includes a motion detection circuit 110, a storage circuit 120 and a processing circuit 130.

The motion detection circuit 110 can be implemented by such as, but not limited to an acceleration sensor (e.g. accelerometer), an angular velocity sensor (e.g. gyroscope), a magnetic induction sensor (e.g. magnetometer) or a combination thereof. The motion detection circuit 110 is configured to detect a human body motion to generate a detection signal SS accordingly. For example, the motion detection circuit 110 can be disposed in a portable electronic device, e.g. smart phone or smart watch that is worn by a user. When the user walks or moves, the motion detection circuit 110 detects movements in multiple axes according to the gravity or the magnetic forces to generate the detection signal SS.

The storage circuit 120 can be any storage device capable of storing data, such as but not limited to a random access memory (RAM), a read only memory (ROM) or a hard drive. It is appreciated that in different embodiments, the storage circuit 120 may include only one or more than one of the storage devices described above to store different types of data. In an embodiment, the storage circuit 120 is configured to store computer executable commands 125.

The processing circuit 130 is electrically coupled to the motion detection circuit 110 and the storage circuit 120, and is configured to retrieve and execute computer executable commands 125 from the storage circuit 120. The computer executable commands 125 includes firmware, driver and related commands of the hardware modules that include such as, but not limited to the motion detection circuit 110 and the storage circuit 120, so as to access the signal or data of the motion detection circuit 110 and the storage circuit 120 to perform operation and further execute the function of the sleep monitoring device 100. The sleep monitoring can be accomplished according to the motion of the user.

The operation of the sleep monitoring device 100 is described in detail in the following paragraphs.

At first, the processing circuit 130 receives the detection signal SS from the motion detection circuit 110. In an embodiment, after the processing circuit 130 receives the detection signal SS, the processing circuit 130 performs data processing on the detection signal SS that includes smoothing, coordinate conversion based on the axes of the motion detection circuit 110 or a combination thereof.

Further, the processing circuit 130 accumulates, in each of a plurality of basic time durations, an activity amount based on a total time length that an activity strength of the detection signal is larger than a predetermined value.

Reference is now made to FIG. 2. FIG. 2 illustrates a diagram of the detection signal SS in a basic time duration TL according to an embodiment of the present invention.

In an embodiment, the processing circuit 130 divides each basic time duration TL into a plurality of sub unit times T1-T10 to determine whether the activity strength of the detection signal SS in each of the sub unit times T1-T10 is larger than the predetermined value VP. The processing circuit 130 accumulates the sub unit times T1-T10 having the activity strength larger than the predetermined value VP to generate the total time length.

For example, the length of the basic time duration TL can be one minute while the basic time duration TL can be divided into 10 sub unit times T1-T10 each having a length of 6 seconds. When the activity strength of the detection signal SS in 5 of the sub unit times (e.g. the sub unit times T2-T4 and T6-T7) is larger than the predetermined value VP, the processing circuit 130 sets the activity amount of the basic time duration TL to be such as, but not limited to 6×5=30. By using the method described above, the processing circuit 130 can keep determining the activity amount of the detection signal SS corresponding to each of the basic time duration TL.

Subsequently, for each of the basic time durations as a target basic time duration, the processing circuit 130 generates statistics based on the activity amount of all the basic time durations in different time ranges around the target basic time duration having different lengths, to generate total activity amounts of the time ranges corresponding to the target basic time duration.

For example, when the length of the basic time duration is one minute, the processing circuit 130 can generate statistics based on the activity amount of the time ranges that are from 1 minute before the target basic time duration to 1 minute after the target basic time duration (totally 3 basic time durations), from 3 minutes before the target basic time duration to 3 minutes after the target basic time duration (totally 7 basic time durations), from 5 minutes before the target basic time duration to 5 minutes after the target basic time duration (totally 11 basic time durations) and from 7 minutes before the target basic time duration to 7 minutes after the target basic time duration (totally 15 basic time durations). The total activity amount of each of the different time ranges can be generated.

The processing circuit 130 can set different activity threshold values each corresponding to one of the time ranges. For each of the basic time durations, whether each of the total activity amounts exceeds one of the activity threshold values is determined to generate an activity scale determination result.

In an embodiment, when the total activity amounts corresponding to the time ranges having the shorter time lengths exceed the corresponding activity threshold values, the corresponding activity scale determination result has a higher activity scale. When only the total activity amounts corresponding to the time ranges having the longer time lengths exceed the corresponding activity threshold values, the corresponding activity scale determination result has a lower activity scale.

For example, when the processing circuit 130 determines that the total activity amount in the time range that is from 1 minute before a target basic time duration to 1 minute after the target basic time duration (totally 3 basic time durations) exceeds the corresponding activity threshold value, the processing circuit 130 generates the activity scale determination result corresponding to such a target basic time duration as “level 4”. When the processing circuit 130 determines that the total activity amount in the time range that is from 3 minute before a target basic time duration to 3 minute after the target basic time duration (totally 7 basic time durations) exceeds the corresponding activity threshold value, the processing circuit 130 generates the activity scale determination result corresponding to such a target basic time duration as “level 3”.

When the processing circuit 130 determines that the total activity amount in the time range that is from 5 minute before a target basic time duration to 5 minute after the target basic time duration (totally 11 basic time durations) exceeds the corresponding activity threshold value, the processing circuit 130 generates the activity scale determination result corresponding to such a target basic time duration as “level 2”. When the processing circuit 130 determines that the total activity amount in the time range that is from 7 minute before a target basic time duration to 7 minute after the target basic time duration (totally 15 basic time durations) exceeds the corresponding activity threshold value, the processing circuit 130 generates the activity scale determination result corresponding to such a target basic time duration as “level 1”. When the processing circuit 130 determines that the total activity amount in the time range that is from 7 minute before a target basic time duration to 7 minute after the target basic time duration (totally 15 basic time durations) does not exceed the corresponding activity threshold value, the processing circuit 130 generates the activity scale determination result corresponding to such a target basic time duration as “level 0”.

The processing circuit 130 determines a sleep status of the user according to the activity scale determination result.

In an embodiment, processing circuit 130 can perform data training based on the actual sleeping condition of the user, to obtain the sleep status corresponding to each of a plurality of combinations of activity scales, e.g. sober, light sleep and deep sleep. Such data can be stored in the storage circuit 120. When the sleep monitoring device 100 operates, the processing circuit 130 determines the sleep status of the user by using such as, but not limited to a lookup table according to the activity scale determination result.

For example, when the activity scale determination result shows that the activity scale is level 4 or level 3, or the activity scale stays in level 2 for a time period longer than a predetermined time period, the processing circuit 130 determines the sleep status to be “sober”. When the activity scale determination result shows that the activity scale stays in level 1 for a time period longer than a predetermined time period, or the activity scale stays in level 1 for a time period longer than a predetermined time period with a certain short time intervals having the activity scale of level 2, the processing circuit 130 determines that the sleep status is “light sleep”. When the activity scale determination result shows that the activity scale stays in level 0 for a time period longer than a predetermined time period, the processing circuit 130 determines that the sleep status is “deep sleep”.

It is appreciated that the method that the processing circuit 130 determines the sleep status based on the activity scale and the length of time of a level that the activity scale stays is merely an example. In other embodiments, the processing circuit 130 may determine the sleep status based on other combinations.

In an embodiment, an actual sleep in a short time period and a sober status that the user stays quiet, e.g. reading, are not easy to be distinguished only according to the activity amount. As a result, besides the activity scale determination result, the processing circuit 130 may determine the sleep status according to such as, but not limited to a status occurrence time spot, a status occurrence time length, a time difference from a neighboring status occurrence time or a combination thereof.

For example, the processing circuit 130 may determine the whether the status occurrence time spot is noon or midnight, whether the status occurrence time length exceeds 2 hours or whether the time difference from the neighboring status occurrence time exceeds 3 hours, so as to distinguish the actual sleep from the sober status that the user stays quiet. When one or more than one determination results shows that the user is in the sleep status, the user is determined to be actually in the sleep status.

As a result, the sleep monitoring device of the present invention can determine the activity amount according to the total time length that has the activity strength of the detection signal larger than the predetermined value. The determination of the sleeps status does not require the accumulation of all the activity amounts. The storage amount and the operation amount required by the sleep monitoring operation can be saved. Further, by using the statistics of the activity amounts in the neighboring time periods, the accuracy of the sleep status determination can be increased.

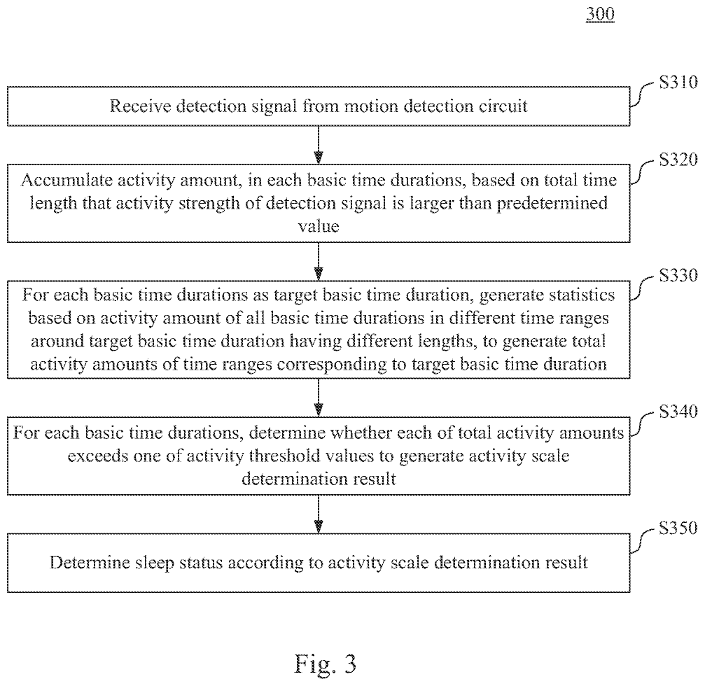

Reference is now made to FIG. 3. FIG. 3 illustrates a flow chart of a sleep monitoring method 300 according to an embodiment of the present invention.

In addition to the apparatus described above, the present disclosure further provides the sleep monitoring method 300 that can be used in such as, but not limited to, the sleep monitoring device 100 in FIG. 1. As illustrated in FIG. 3, an embodiment of the sleep monitoring method 300 includes the following steps.

In step S310, the detection signal SS from the motion detection circuit 110 is received by the processing circuit 130.

In step S320, the activity amount, in each of the basic time durations, is accumulated based on the total time length that the activity strength of the detection signal SS is larger than the predetermined value by the processing circuit 130.

In step S330, for each of the basic time durations as the target basic time duration, statistics is generated based on the activity amount of all the basic time durations in different time ranges around the target basic time duration having different lengths, to generate total activity amounts of the time ranges corresponding to the target basic time duration by the processing circuit 130.

In step S340, for each of the basic time durations, whether each of the total activity amounts exceeds one of a plurality of activity threshold values is determined to generate the activity scale determination result by the processing circuit 130, wherein each of the activity threshold values corresponds to one of the time ranges.

In step S350, the sleep status is determined according to the activity scale determination result by the processing circuit 130.

It is appreciated that the embodiments described above are merely an example. In other embodiments, it should be appreciated that many modifications and changes may be made by those of ordinary skill in the art without departing, from the spirit of the disclosure.

In summary, the sleep monitoring device and the sleep monitoring method of the present invention can determine the activity amount according to the total time length that has the activity strength of the detection signal larger than the predetermined value. The determination of the sleeps status does not require the accumulation of all the activity amounts. The storage amount and the operation amount required by the sleep monitoring operation can be saved. Further, by using the statistics of the activity amounts in the neighboring time periods, the accuracy of the sleep status determination can be increased.

The aforementioned descriptions represent merely the preferred embodiments of the present invention, without any intention to limit the scope of the present invention thereto. Various equivalent changes, alterations, or modifications based on the claims of present invention are all consequently viewed as being embraced by the scope of the present invention.

What is claimed is:

1. A device, comprising:
a motion detection circuit configured to detect a human body motion to generate a detection signal accordingly;
a storage circuit; and
a processing circuit electrically coupled to the motion detection circuit and the storage circuit, and configured to retrieve and execute computer executable commands to execute steps for sleep monitoring comprising:
receiving the detection signal from the motion detection circuit;
accumulating, in each of a plurality of basic time durations, an activity amount based on a total time length that an activity strength of the detection signal is larger than a predetermined value;
for each of the basic time durations as a target basic time duration, generating statistics based on the activity amount of all the basic time durations in different time ranges around the target basic time duration having different lengths, to generate total activity amounts of the time ranges corresponding to the target basic time duration;
for each of the basic time durations, whether each of the total activity amounts exceeds one of a plurality of activity threshold values is determined to generate an activity scale determination result, wherein each of the activity threshold values corresponds to one of the time ranges; and
determining a sleep status according to the activity scale determination result.

2. The device of claim 1, wherein the sleep monitoring method further comprises:
determining the sleep status according to the activity scale determination result, a status occurrence time spot, a status occurrence time length, a time difference from a neighboring status occurrence time or a combination thereof.

3. The device of claim 1, wherein when the total activity amounts corresponding to the time ranges having the shorter time lengths exceed the corresponding activity threshold values, the corresponding activity scale determination result has a higher activity scale;
when only the total activity amounts corresponding to the time ranges having the longer time lengths exceed the corresponding activity threshold values, the corresponding activity scale determination result has a lower activity scale.

4. The device of claim 1, wherein the sleep monitoring method further comprises:
dividing each of the basic time durations into a plurality of sub unit times to determine whether the activity strength of the detection signal in each of the sub unit times is larger than the predetermined value; and
accumulating the sub unit times having the activity strength larger than the predetermined value to generate the total time length.

5. The device of claim 1, wherein the sleep monitoring method further comprises:
performing data processing on the detection signal that comprises smoothing, coordinate conversion or a combination thereof.

6. A method, comprising:
detecting a human body motion by a motion detection circuit to generate a detection signal accordingly;
receiving the detection signal from the motion detection circuit by a processing circuit;
accumulating an activity amount, in each of basic time durations, based on a total time length that an activity strength of the detection signal is larger than a predetermined value by the processing circuit;
for each of the basic time durations as a target basic time duration, generating statistics based on the activity amount of all the basic time durations in different time ranges around the target basic time duration having different lengths, to generate total activity amounts of the time ranges corresponding to the target basic time duration by the processing circuit;
for each of the basic time durations, whether each of the total activity amounts exceeds one of a plurality of activity threshold values is determined to generate an activity scale determination result by the processing circuit, wherein each of the activity threshold values corresponds to one of the time ranges; and
determining a sleep status according to the activity scale determination result by the processing circuit.

7. The method of claim 6, further comprising:
determining the sleep status according to the activity scale determination result, a status occurrence time spot, a status occurrence time length, a time difference from a neighboring status occurrence time or a combination thereof by the processing circuit.

8. The method of claim 6, wherein when the total activity amounts corresponding to the time ranges having the shorter time lengths exceed the corresponding activity threshold values, the corresponding activity scale determination result has a higher activity scale;
when only the total activity amounts corresponding to the time ranges having the longer time lengths exceed the corresponding activity threshold values, the corresponding activity scale determination result has a lower activity scale.

9. The method of claim 6 further comprising:
dividing each of the basic time durations into a plurality of sub unit times to determine whether the activity strength of the detection signal in each of the sub unit times is larger than the predetermined value by the processing circuit; and
accumulating the sub unit times having the activity strength larger than the predetermined value to generate the total time length by the processing circuit.

10. The method of claim 6 further comprising:
performing data processing on the detection signal that comprises averaging, coordinate conversion or a combination thereof by the processing circuit.
